(19) United States
(12) Patent Application Publication
(10) Pub. No.: US 2026/0000582 A1
(43) Pub. Date: Jan. 1, 2026

(54) IONTOPHORESIS AND SONOPHORESIS DUAL TREATMENT

(71) Applicant: L'Oreal, Paris (FR)

(72) Inventors: Ayoub HBAICH, Franklin Park, NJ (US); Rafael FELICIANO, New Providence, NJ (US)

(73) Assignee: L'Oreal, Paris (FR)

(21) Appl. No.: 18/756,840

(22) Filed: Jun. 27, 2024

(51) Int. Cl. A61K 8/02; A61N 1/04; A61N 7/00; A61Q 19/00

(52) U.S. Cl. CPC A61K 8/0204; A61N 1/0448; A61N 7/00; A61Q 19/00; A61K 2800/83; A61K 2800/874; A61N 2007/0034

(57) ABSTRACT

Systems and methods for application of a composition to the skin of a subject using electroporation, iontophoresis, and in at least some instances, sonophoresis to facilitate penetration of the skin by one or more compounds of the composition, such as one or more drugs. A system includes an applicator capsule that includes a rollerball configured for a rolling contact with the portion of skin of the subject and a plurality of electrodes and/or ultrasound transducers that are configured for a transmission of an electrical current and/or ultrasound through the portion of skin of the subject. A formula pod of the system is reversibly attachable to the body and is configured to hold and release the composition therefrom for delivery of the composition from the formula pod to the portion of skin.

### SUMMARY

In an aspect, the disclosure provides a system for application of a composition to a portion of skin of a subject, the system comprising: an applicator capsule, comprising: a rollerball configured for a rolling contact with the portion of skin of the subject and a plurality of electrodes configured for a transmission of an electrical current through the portion of skin of the subject, a reservoir configured to hold and release the composition therefrom, and an ultrasound transducer configured to transmit ultrasound for sonophoresis; and a body, configured for reversible attachment of the applicator capsule thereto, and comprising a contact-less piston configured to expel the composition from the reservoir; wherein one or more apertures of the applicator capsule fluidly connect the interior of the applicator capsule to an exterior of the applicator capsule for delivery of the composition from the reservoir to the portion of skin.

In embodiments, the system further comprises control circuitry configured to electronically control the at least one operation of the contact-less piston.

In embodiments, the system further comprises control circuitry operably connected to and configured to receive a signal from a microchip of the reservoir that corresponds with one or more compounds of the composition, wherein the control circuitry electronically controls the transmission of the electrical current, based on a program selected by the control circuitry based at least in part on the signal, for delivery of the one or more compounds to the portion of skin of the subject.

In embodiments, the one or more compounds of the composition comprises a large molecule, a small molecule, or both, and wherein the program configures the control circuitry for the transmission of the electrical current based on the signal.

In embodiments, the contact-less piston is responsive to an activation of an electromagnet and the at least one operation comprises activation of the electromagnet for movement of the contact-less piston and displacement of the composition from the formula pod.

In embodiments, the electrical current comprises electroporesis of at least one compound of the composition to the portion of skin.

In embodiments, the electrical current comprises iontophoresis of at least one compound of the composition through the portion of skin.

In embodiments, the iontophoresis delivers a large molecule of the composition, a small molecule of the composition, or both, through a dermal layer of the portion of skin.

In embodiments, the electrical current activates the ultrasound transducer for sonophoresis.

In embodiments, the sonophoresis creates one or more microchannels in a dermal layer of the portion of skin.

In embodiments, the ultrasound for sonophoresis is transmitted at a frequency in the range of 100 kilohertz to 1 megahertz.

In an aspect, the disclosure provides a method for administering a composition to a portion of skin of a subject, the method comprising: activating, with control circuitry of the system, an electromagnet of the system to move a contact-less piston of the system and displace at least the portion of the composition from a reservoir for expulsion of at least the portion of the composition from one or more apertures of the applicator capsule, responsive to receiving, with control circuitry of the system, a signal from a microchip of a reservoir of the system, wherein the reservoir contains at least a portion of the composition therein and the signal corresponds with one or more compounds of the composition in the reservoir; and transmitting, with control circuitry of the system, an electrical current through the portion of skin of the subject, wherein transmission of the electrical current is based on a program selected by the control circuitry based at least in part on the signal.

In embodiments, the electrical current comprises electroporesis of at least one compound of the composition to the portion of skin.

In embodiments, the electrical current comprises iontophoresis of at least one compound of the composition through the portion of skin.

In embodiments, the electrical current activates an ultrasound transducer for sonophoresis.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

FIG. 1 shows a perspective view of an example applicator capsule, according to aspects of the disclosure.

FIG. 2 shows a cross-sectional view of an example applicator capsule, according to aspects of the disclosure.

FIG. 3 shows a cross-sectional view of an example applicator capsule, according to aspects of the disclosure.

FIG. 4 shows a perspective view of an example system, according to aspects of the disclosure.

FIG. 5 shows a perspective view of an example system, according to aspects of the disclosure.

FIG. 6 shows an example formula pod and system, according to aspects of the disclosure.

FIG. 7 shows an example component diagram of an example system, according to aspects of the disclosure.

FIG. 8 shows an example program selection of an example system, according to aspects of the disclosure.

FIG. 9 shows an example method of use of an application system to apply a formula to a portion of an individual's skin, according to aspects of the disclosure.

FIG. 10 shows a perspective view of an example applicator capsule in an exploded configuration, showing an example piston and piston assembly, according to aspects of the disclosure.

FIG. 11 shows a cross-sectional view of an example applicator capsule showing an example piston and piston assembly in a first position, according to aspects of the disclosure.

FIG. 12 shows a cross-sectional view of an example applicator capsule showing an example piston and piston assembly in a second position, according to aspects of the disclosure.

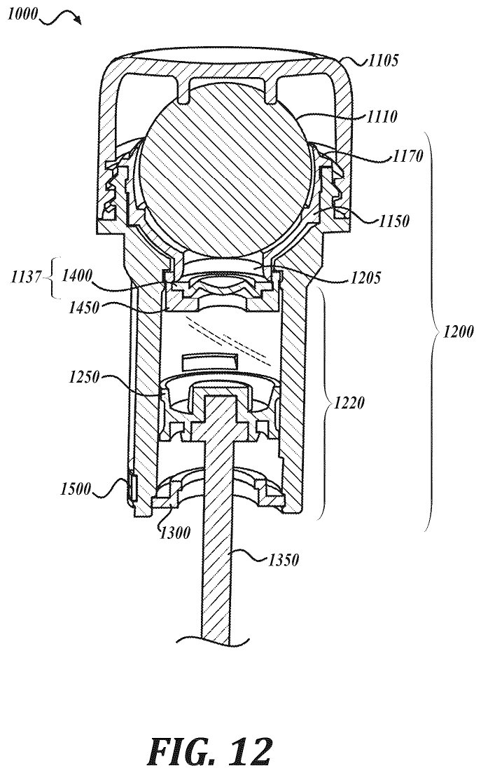

TABLE 1
Drawings elements and descriptions.

| Numeric Label | Description |
|---|---|
| 100 | Applicator capsule |
| 110 | Roller ball |
| 111 | Roller ball aperture |
| 112 | Formula pod |
| 113 | Formula pod microchip |
| 120 | Contact face |
| 121 | Electrode aperture |
| 130a | Electrode |
| 130b | Electrode |
| 130c | Electrode |
| 130d | Electrode |
| 140 | Insert |
| 141 | Adapter |
| 142a | Locking groove |
| 142b | Locking groove |
| 150 | Electrode contact |
| 160 | Adapter |
| 161a | Aperture (outer portion) |
| 161b | Aperture (inner portion) |
| 162 | Interior |
| 200 | Body |
| 201 | Insert attachment |
| 202 | Body surface |
| 203 | Reservoir attachment |
| 204 | Reservoir |
| 205a | Power button/control |
| 205b | Eject button/control |
| 206 | Status indicator |
| 207 | Aperture |
| 208 | Electrical connection |
| 300 | Application system |
| 400 | Application system |
| 500a | Radiofrequency (RF) warming/ultrasound |
| 500b | Electroporesis and/or iontophoresis |
| 600 | Application system diagram |
| 601 | Applicator capsule |
| 602 | Microcurrent (μc) |
| 603 | Computational circuitry |
| 604 | User input |
| 605 | Pulse |
| 606 | Pulse |
| 607 | High voltage |
| 608 | High frequency |
| 609 | Electrode (electroporation and/or microcurrent) |
| 610 | Piezoelectric ultrasound transducer(s) (ultrasound |
| | warming) |
| 611 | Circuitry (electroporation) |
| 612 | Circuitry (microcurrent) |
| 700 | Application system diagram |
| 701 | Applicator capsule |
| 702 | Microcurrent (μc) |
| 703 | Microcurrent check |
| 704 | Large molecules |
| 705 | Small molecules |
| 706 | Electroporation |
| 707 | Microcurrent |
| 800 | Method of use of application system to apply formula to |
| | a portion of an individual's skin |
| 801 | Start |
| 802 | Attach applicator |
| 803 | Identify formula |
| 804 | Move piston and formula |
| 805 | Dispense formula |
| 806 | Administer treatment |
| 807 | Apply formula |
| 808 | Finish |
| 900 | System |
| 1000 | Capsule |
| 1105 | Cap |
| 1110 | Rollerball |
| 1137 | Solution regulator |
| 1170 | Rollerball retainer |
| 1150 | Fitment |
| 1205 | Well |
| 1370 | Solution regulator |
| 1200 | Body |
| 1220 | Reservoir |
| 1250 | Piston |
| 1300 | Plug |
| 1350 | Push rod |
| 1400 | Check valve |
| 1450 | Check valve holder |
| 1500 | Authentication chip |

The foregoing aspects and many of the attendant advantages of this disclosure will become more readily appreciated as the same become better understood by reference to the following detailed description, when taken in conjunction with the accompanying drawings.

### DETAILED DESCRIPTION

Iontophoresis is a non-invasive technique wherein a physiologically acceptable amount of electric current (e.g., up to about 0.5 mA/cm² or typically 10 V or less) is used to facilitate transdermal delivery of charged and/or neutral molecules. Iontophoresis does not tend to disrupt the skin barrier in promoting transdermal flux, and acts directly on one or more compounds of a composition applied to the skin to deliver the compound into deeper layers of the skin. Electroporation applies a higher voltage (typically about 100 V) pulse for a very short (microseconds to milliseconds) duration to permeabilize the skin. Sonophoresis is the use of ultrasound to drive molecules into and across skin. Ultrasound for transdermal permeation typically includes sonophoresis at lower frequencies, including in the range of about 20 to 100 kHz, for example.

These techniques can potentially enable drug delivery across the skin and expand the scope of transdermal delivery to include not only small molecules but large molecules as well, such as proteinaceous drugs of the biotechnology industry. However, the potential of these techniques has not been fully realized due to the skin maintaining a barrier that restricts or prevents passage of the molecules therethrough. In addition, iontophoresis and electroporesis systems, and sonophoresis systems, are typically configured for use with distinct classes of molecules that are delivered with different electrical currents and/or sonophoresis frequencies. A therapeutic composition that includes one or more biologically active molecules that are from distinct classes may involve both iontophoresis/electroporesis systems as well as sonophoresis systems, and this is an inconvenience for the user or clinician and can result in lower adherence to a treatment plan or further health complications due to inadequate treatment.

Accordingly, there is a need for intradermal and transdermal therapeutic delivery systems that are configured for iontophoresis/electroporesis and sonophoresis, that can more effectively deliver a wide range of molecule types to and through the skin, and that are configured to warm compositions during use to relax the skin layers and widen the gaps between skin cells for more effective delivery of compounds therein. The present disclosure addresses these and other long-felt and unmet needs in the art.

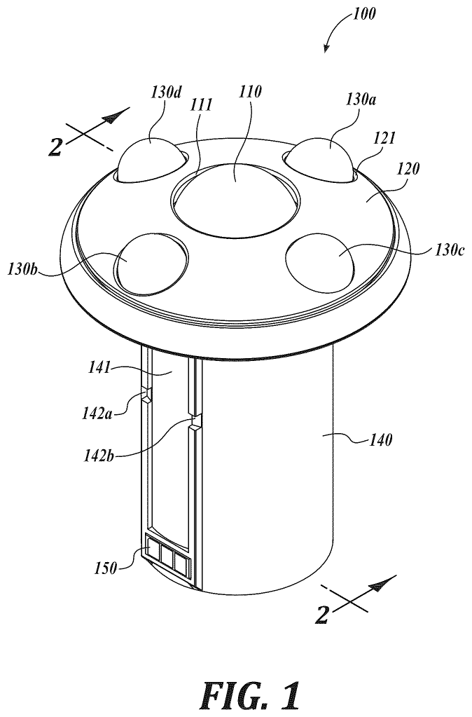

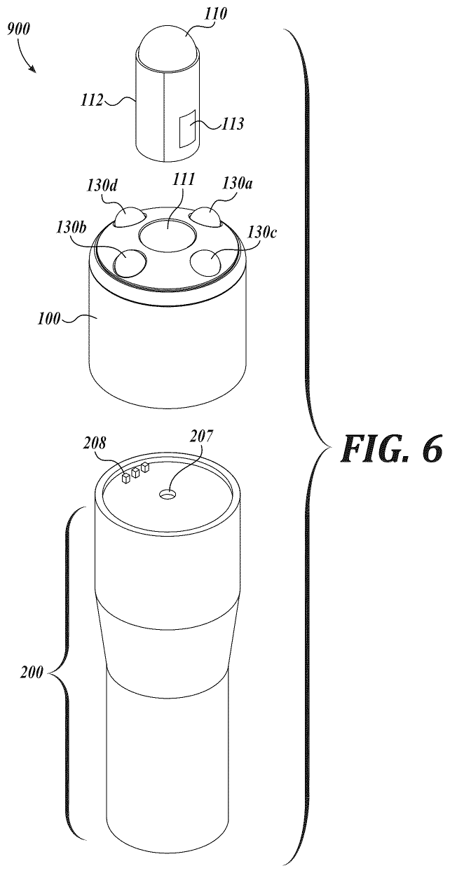

As shown at FIGS. 1-3, an example applicator capsule 100 comprises a roller ball 110, positioned within a roller ball aperture 111, that makes a rolling contact with the skin of a subject and rolls across the skin when a contact face 120 is placed adjacent to the skin during use. The roller ball 110 can be an element of a formula pod 112, as shown by way of non-limiting example at FIG. 6. In the shown embodiment, the applicator capsule 100 comprises a plurality of electrodes, including electrodes 130a, 130b, 130c, and 130d, configured for a transmission of an electrical current through the portion of skin of the subject during use. While four electrodes are included in the example embodiment, other amounts of electrodes can be implemented, in embodiments, without departing from the scope and spirit of the disclosure. Examples of alternative amounts of electrodes include but are not limited to one electrode, two electrodes, three electrodes, five electrodes, six electrodes, seven electrodes, eight electrodes, nine electrodes, ten electrodes, or more.

In the shown embodiment, the applicator capsule 100 comprises an insert 140 that is configured to be inserted into a body of the system for attachment of the applicator capsule 100 to the body. The shown insert 140 includes an adapter 141 fitted with locking grooves 142a and 142b, which can slidingly and reversibly lock to the body when the applicator capsule 100 is attached thereto. An electrode contact 150 is operably connected to the electrodes 130a, 130b, 130c, and 130d, by way of one or more electrical connections, for delivery of the electrical current from a power source through the electrode contact 150 to the electrodes 130a, 130b, 130c, and 130d. While the shown insert 140 includes a substantially spherical cross sectional shape with a flat portion for the adapter 141, other cross sectional shapes can be implemented without departing from the scope and spirit of the disclosure, with the understanding that the insert 140 would need to be able to be secured to the body of the system, and the body can optionally include a correspondingly shaped cavity for receipt of the insert 140 therein.

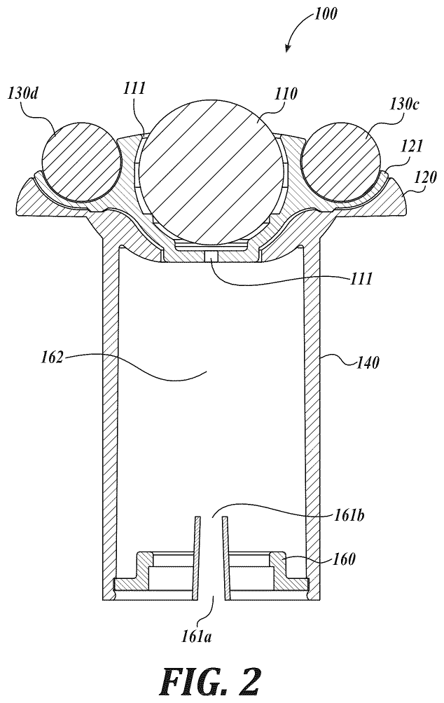

In the embodiment of FIG. 2, an interior 162 of the applicator capsule 100 is operably connected to an interior of the body of the system by way of an aperture 161a at an outer portion of the applicator capsule 100 and an aperture 161b at an inner portion of the applicator capsule 100. In example embodiments, a piston can pass through the aperture 161a for displacement of the composition from the formula pod. In the shown embodiment, the interior 162 of the applicator capsule 100 occupies a substantial or majority of the volume of the applicator capsule 100, however, in other embodiments, the interior 162 can occupy a lesser or minority of the volume of the applicator capsule 100 without departing from the scope and spirit of the disclosure.

The insert 140 can include an interface, such as an adapter 160, that interfaces with a correspondingly shaped interface within the interior of the body of the system to form a fluidic seal to prevent leakage of a composition therefrom. During use of the shown example system, the composition flows from the interior 162 of the applicator capsule 100, until it reaches the roller ball aperture 111. The composition flows through the roller ball aperture 111 and out of the applicator capsule 100, where it contacts the roller ball 110 and the portion of skin of the subject during application.

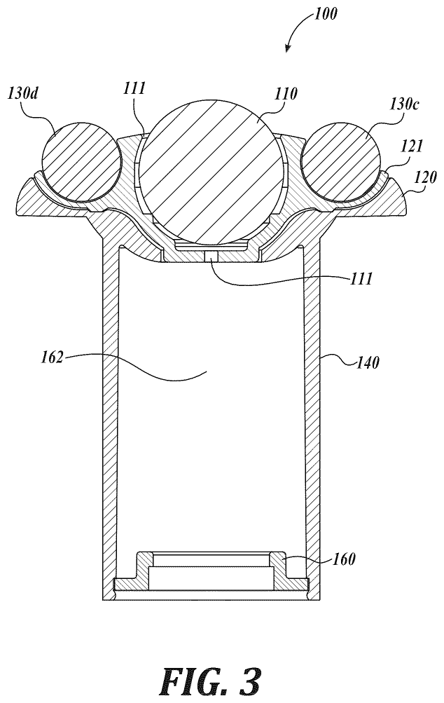

In the embodiment of FIG. 3, an interior 162 of the applicator capsule 100 is not fluidly connected to an interior of the body of the system, and does not include apertures as shown at FIG. 2 (i.e., apertures 161a, 161b). Instead, in the embodiment of FIG. 3, the interior 162 can comprise a composition therein and the adapter 160 can be coupled, on a lower portion thereof, to a piston. With an upward movement of the piston, the adapter 160 can slide upward within the interior 162 and compress the composition therein, such that the composition is expelled through roller ball aperture 11 and out of the applicator capsule 100, where it contacts the roller ball 110 and the portion of skin of the subject during application. In embodiments, after use, the applicator capsule 100 can be removed and cleaned for reuse.

As shown at FIG. 6, an example system 900 for application of a composition to a portion of skin of a subject comprises an applicator capsule 100, comprising a plurality of electrodes (130a, 130b, 130c, 130d) configured for a transmission of an electrical current through the portion of skin of the subject, and a body 200, configured for reversible attachment of the applicator capsule 100 thereto, and a formula pod 112 reversibly that comprises a rollerball 110 configured for a rolling contact with the portion of skin of the subject. The formula pod 112 is attachable to the applicator capsule 100 and is configured to hold and release the composition, from a reservoir within the formula pod 112, based on at least one operation of a contact-less piston of the body 200 for delivery of the composition from the reservoir of the formula pod 112 to the portion of skin.

As such, the roller ball 110 can be an element of a formula pod 112, as shown by way of non-limiting example at FIG. 6. An example formula pod 112 of system 900, according to embodiments, comprises a composition for application to the skin of the subject therein, and can be inserted into an applicator capsule 100 and thereby operably connected to a body 200 of the system 900. In the shown embodiment, a piston can extend through an aperture 207 of the body 200 and an interior of the applicator capsule 100, and can mechanically expel the formula from the formula pod 112 with a movement of the piston. A power source of the body 200 can be operably connected to the applicator capsule 100 by way of an electrical connection 208, which can be configured to electrically connect with a corresponding electrical connection of the applicator capsule 100. In the shown embodiment, a formula pod microchip 113 is included and configured to enable identification of the composition in the formula pod, and/or one or more compounds thereof, for selection of a program for electroporation and/or iontophoresis application of the composition, as described herein. Since the formula pod 112 is easily removable from the device, it can be exchanged for alternative formula pods 112, e.g., alternate compositions. In the shown embodiment, the body 200 of the device may not include a reservoir as in other implementations, since the composition can be supplied by the formula pod 112, as shown.

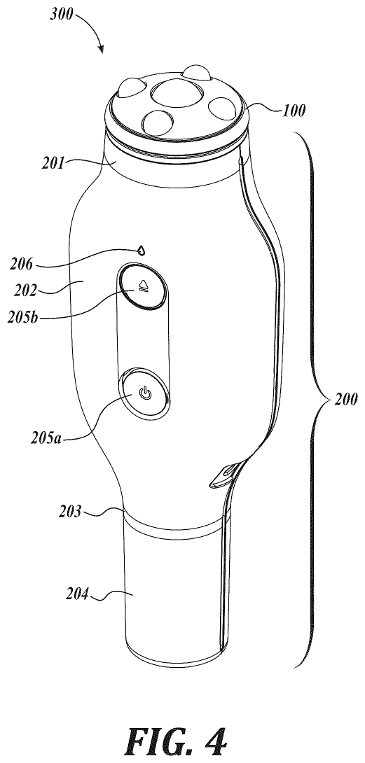

As shown at FIG. 4, an example application system 300 for application of a composition to a portion of skin of a subject can include a form factor that includes a body 200 with a body surface 202 that is shaped and/or configured, e.g., ergonomically shaped, to be gripped by a user for operation of the system 300. In the shown embodiment, an applicator capsule 100 is attachable to the body 200 by way of an insert attachment 201. The insert attachment 201 can include a snap-on attachment, for example, such that the adapter 141 (of FIG. 1) can be inserted into the body 200 and secured in place by the snap-on attachment of the insert attachment 201, or alternatively, by way of locking grooves (142a, 142b of FIG. 1) being locked in place. Other attachment mechanisms can be implemented for the insert attachment 201 without departing from the scope and spirit of the disclosure.

In embodiments, body 200 comprises a power source (e.g., battery or rechargeable battery) operably connected to an actuator by way of one or more electrical couplings. The actuator can mechanically actuate a piston of the system to transmit a portion of a composition of a reservoir 304 of the system, from the reservoir 204 through the body 200 and to the applicator capsule 100. The power source can be electrically coupled to the electrodes (130a, 130b, 130c, 130d of FIG. 1) for transmission of electric current to the electrodes based on a program of the system, for example.

In embodiments, the system 300 can include a reservoir 204 attachable to the body 200 by a reservoir attachment 203. The reservoir 204 is reversibly attachable to the body 200 and can be attached, such as by way of a snap-on attachment or other attachment mechanism, and removed by way of reversal of the snap-on attachment or other attachment mechanism. The reservoir 204 is configured to hold the composition therein, and release the composition therefrom, such that the composition flows through an interior of the body to an interior of the applicator capsule (e.g., 162 of FIG. 2) based on at least one operation of a piston or contact-less piston of the body. With movement of the composition through the interior of the body and the interior of the applicator capsule, and out the roller ball aperture 111, the composition can effectively flow from the reservoir 204 through the body 200 of the system 300 and out the applicator capsule 100, where it contacts the skin and is ready to be rollingly applied to the skin and electrically transmitted to and/or through one or more layers of the skin, as described herein.

In embodiments, the system 300 can be controlled at least in part by a user through control circuitry, which can include a power button 205a and/or an eject button 205b operably connected to the control circuitry by way of one or more electronic connections. In the shown embodiment, the power button 205a can be activated by a user to power on the system 300 or to activate a functionality of the system 300, such as an operation of the contact-less piston for expulsion of the composition, or a cleaning solution or liquid, from the system 300. In the shown embodiment, the eject button 205b can be activated by a user to eject the reservoir 204 from the body 200 of the system 300, for example, for replacement of the reservoir 204 and/or cleaning or maintenance of the system 300 or a component thereof. In the shown embodiment, a status indicator 206 is included and is operably connected to the control circuitry, or other circuitry of the system 300, for indicating a status of the system 300 to the user. The status indicator 206 can indicate status of one or more components of the system, such as charge level of a rechargeable battery of the system, fill level of the reservoir, need for cleaning of the system or a component thereof, and the like.

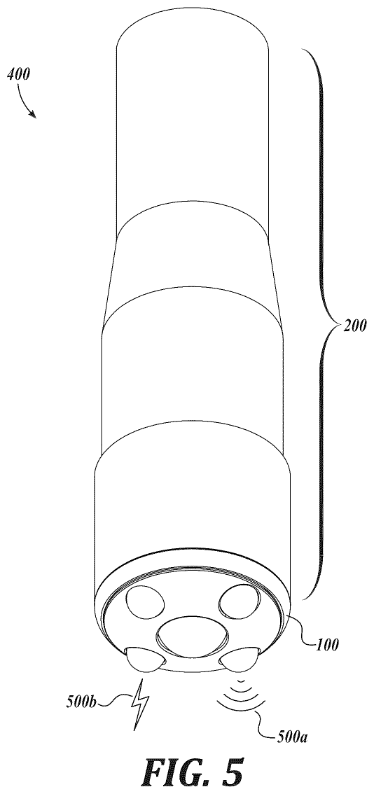

FIG. 5 shows another example embodiment of an application system 400 for application of a composition to a portion of skin of a subject. The system 400 includes the body 200 and can include, in various aspects, an applicator capsule 100, a rollerball configured for a rolling contact with the portion of skin of the subject, and a plurality of electrodes. The system 400 can include one or more electrodes configured or configurable for a radiofrequency (RF) warming or ultrasound electrical transmission 500a, as well as one or more electrodes configured or configurable for an electroporesis and/or iontophoresis electrical transmission 500b, as in the shown embodiment. The electrodes can be operably connected to control circuitry of the system 400, as well as a power source of the system, such as a rechargeable battery, for the generation of one or more current patterns. The one or more current patterns can be at least a part of a program for delivery of one or more molecules of the composition of the reservoir to the portion of skin of the subject. The program can be selected, by the control circuitry, based at least in part on the composition (or an identifying characteristic thereof), as described herein.

In embodiments, a formula pod can include a microchip that corresponds to one or more compounds of a composition within the formula pod. The system can select, based on an identifying characteristic of the composition obtainable by the system from the microchip, a program for administration of therapeutic electrical current(s). Characteristics of the composition can include one or more molecular weights of one or more compounds, one or more ionic states of one or more compounds at a given pH and ionic strength of the composition, or the like. For example, larger molecules of a composition may require a stronger current to be applied to effectively carry the molecules to and through one or more layers of the skin. In embodiments, a user can control one or more modes of operation, or programs, of the system to increase or decrease current based on user preference.

In embodiments, a plurality of energy modes, e.g., three modes, can be implemented or used during operation of the system. For example, electroporesis, iontophoresis, sonophoresis, or any combination thereof can be used to achieve increased penetration and efficacy compared to individual energy modes used separately. For example, a program could start with electroporesis (e.g., creation of channels in the skin barrier using a pulsing current), then iontophoresis can be applied constructively (e.g., repulsion of molecules through the skin barrier and opened channels), while sonophoresis can be applied concurrent with one or more such electrical treatments to increase active absorption, which can be controlled by a user input (e.g., selection of heat or warmth level or intensity) or based on the formula within the reservoir. In embodiments, one energy mode is used in a program. In embodiments, two energy modes are used, in parallel or in sequence, in a program. In yet further embodiments, three energy modes are used, in parallel or in sequence, in a program.

FIG. 6 shows an example formula pod 112 and system 900, according to aspects of the disclosure. In the shown embodiment, a formula pod 112, which comprises a composition for application to the skin of the subject therein, can be inserted into an applicator capsule 100 and thereby operably connected to a body 200 of the system 900. In the shown embodiment, a piston can extend through an aperture 207 of the body 200 and an interior of the applicator capsule 100, and can mechanically expel the formula from the formula pod 112 with a movement of the piston. A power source of the body 200 can be operably connected to the applicator capsule 100 by way of an electrical connection 208, which can be configured to electrically connect with a corresponding electrical connection of the applicator capsule 100. In the shown embodiment, a formula pod microchip 113 is included and configured to enable identification of the composition in the formula pod, and/or one or more compounds thereof, for selection of a program for electroporation and/or iontophoresis application of the composition, as described herein. Since the formula pod 112 is easily removable from the device, it can be exchanged for alternative formula pods 112, e.g., alternate compositions. In the shown embodiment, the body 200 of the device may not include a reservoir as in other implementations, since the composition can be supplied by the formula pod 112, as shown.

Accordingly, in aspects, the disclosure provides a “smart” system comprising circuitry configured to perform all or part of a method, including but not necessarily limited to control of expulsion of the composition from the system, monitoring of a level of the composition within the reservoir, monitoring of a level of charge of the power source, and the like. In embodiments, circuitry of a device is configurable with a processor and processor-executable instructions stored on a non-transitory machine-readable medium of the device. In embodiments, a device includes a software application configured to perform all or part of one or more methods or processes of the disclosure, in any order or combination. However, in embodiments, a device includes dedicated hardware circuitry. Further configuration of circuitry of the device can include wireless communication or networking circuitry, for example, circuitry configured for a wireless connection, such as a Bluetooth® connection, a Bluetooth® low energy (BLE) connection, and/or a Wi-Fi® connection, and/or a wired connection. The networking circuitry, in combination with other circuitry of the computational device, can be used to request, retrieve, and/or receive data from a computational device or a remote server, for example. In embodiments, the device can be operated with use of a computational device, such as a smartphone or personal computational device, that can be operated by a user via a graphical user interface, as known in the art. In embodiments, the circuitry can include operable connection of one or more sensors with the processor, or other circuitry, for performing logic operations and/or methods based on data received from the one or more sensors, for example.

As shown at FIG. 6, an example system 900 for application of a composition to a portion of skin of a subject comprises an applicator capsule 100, comprising a plurality of electrodes (130a, 130b, 130c, 130d) configured for a transmission of an electrical current through the portion of skin of the subject, and a body 200, configured for reversible attachment of the applicator capsule 100 thereto, and a formula pod 112 reversibly that comprises a rollerball 110 configured for a rolling contact with the portion of skin of the subject. The formula pod 112 is attachable to the applicator capsule 100 and is configured to hold and release the composition therefrom based on at least one operation of a contact-less piston of the body 200 for delivery of the composition from the interior of the formula pod 112 to the portion of skin.

In the shown embodiment, the system 900 includes control circuitry configured to electronically control at least one operation of the contact-less piston for expulsion of the composition from the formula pod. The shown embodiment also includes control circuitry operably connected to and configured to receive a signal from a microchip 113 of the formula pod 112 that corresponds with one or more compounds of the composition in the formula pod 112. The control circuitry can electronically control the transmission of the electrical current, based on a program selected by the control circuitry based at least in part on the signal, for delivery of the one or more compounds to the portion of skin of the subject. This can be accomplished by iontophoresis and/or electroporation, sonophoresis, or any combination thereof.

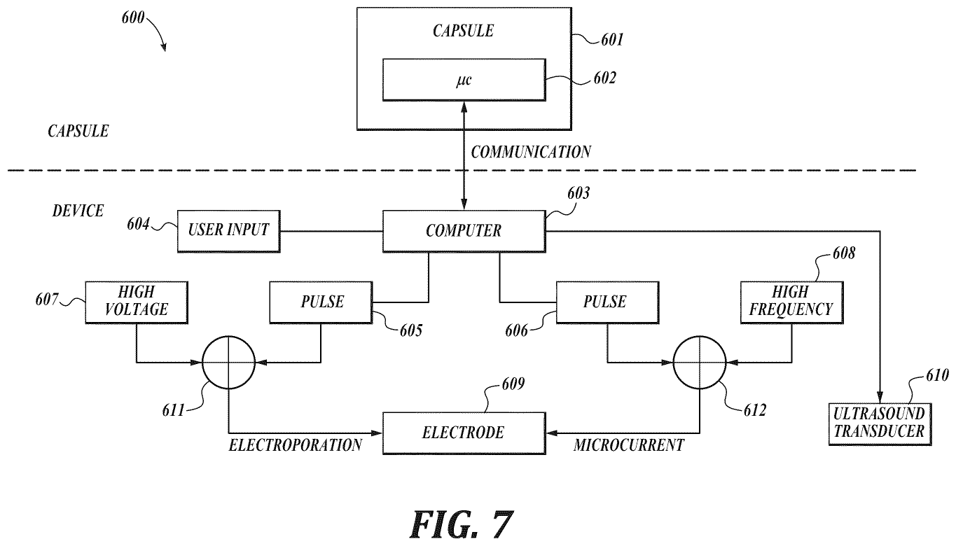

As shown at FIG. 7, an example system 600 includes an applicator capsule 601 operably connectable to the body, by way of one or more operable connections, e.g., physical, fluid, and electronic connections, for electronic and fluid communication between the capsule 601 and the body of the device. The applicator capsule 601 includes one or more electrodes thereon that are operably connected to control circuitry of the system 600 for delivery of a microcurrent 602, e.g., for transmission of electroporation current, iontophoresis current, warming ultrasound, or any combination thereof, to the portion of skin of the subject.

In the shown embodiment, the system 600 includes a computer or computational circuitry 603, which can comprise, control, and/or direct control circuitry, or other circuitry of the system 600, for carrying out one or more operations of the system 600. Computational circuitry 603 can include hardware circuitry, processor circuitry, or both, configured for execution of logic operations, such as activation and deactivation of an electromagnet for movement of the contact-less piston and expulsion of the composition from the system 600, selection and execution of one or more programs for delivery of microcurrent (e.g., electroporation, iontophoresis, sonophoresis e.g., by activation of one or more piezoelectric spherical ultrasound transducers for warming), monitoring charge status of a power source, monitoring percentage of the composition utilized in the formula pod, and the like. Computational circuitry 603 can comprise a non-transitory computer-readable medium having stored thereon instructions which, when executed by one or more processors, configure one or more processors for performance of all or part of a method or operation of the disclosure, in whole or in part, with the steps being in any order. The system 600 can be configured to accept user input 604 from a user, which can include an activation or deactivation signal (e.g., with use of a power button), a status inquiry signal (e.g., with use of a power button or other control element), or the like.

Computational circuitry 603 controls, coordinates, directs, or otherwise enables transmission of microcurrents as part of execution of programs for administration of compositions to the skin. In embodiments, computational circuitry 603 enables transmission of one or more pulses (605, 606) for delivery of current that is high voltage 607 and/or high frequency 608, by way of electroporation circuitry 611 and/or microcurrent circuitry 612, and in at least some instances, sonophoresis with one or more ultrasound transducers 610. The computational circuitry 603 also controls, coordinates, directs, or otherwise enables transmission of current for sonophoresis by way of ultrasound transducer circuitry 610. An electrode 609 can be configured and used for electroporesis, iontophoresis, or both (sequentially and/or simultaneously), and the ultrasound transducer circuitry 610 can be configured and used for sonophoresis.

In embodiments, computational circuitry 603 can be configured to electronically control at least one operation of the contact-less piston. For example, computational circuitry 603 can, as part of execution of at least one program for application of a composition, deliver an electric current to an electromagnet of the system to activate the electromagnet and attract or repulse the contact-less piston, which can be magnetic and/or operably connected to a magnet, for movement of the contact-less piston and displacement of the composition in the body of the system 600.

In embodiments, computational circuitry 603 can be operably connected to and configured to receive a signal from a microchip of the reservoir that corresponds with one or more compounds of the composition in the reservoir. The control circuitry can thereby identify what one or more compounds are in the composition that are to be delivered to and/or through the skin, and can electronically control the transmission of the electrical current, based on a program selected by the control circuitry, for delivery of the one or more compounds to the portion of skin of the subject. For example, FIG. 7 shows an example program selection of an example system 700. In the shown system, a capsule 701 can be attached to the body of the device for delivery of a microcurrent 702 based on an identification of the composition or a compound thereof. Control circuitry of the device can receive a signal from the microchip, through a wired and/or wireless transmission, for example, a radiofrequency identification (RFID) process, that corresponds to identity of one or more compounds of the composition, or the composition as a whole. The system 700 can select a program for delivery of microcurrent 703 based on the identity of the one or more compounds or the composition as a whole. For example, if one or more large molecules 704 are present in the composition as active ingredients to be delivered into the skin, the system can select a program comprising electroporation 706 of the large molecules 704. If one or more small molecules 705 are present in the composition as active ingredients to be delivered into the skin, the system can select a program comprising microcurrent 707, e.g., iontophoresis. In embodiments, a program that includes only electroporation 706 (and not microcurrent 707) is selected. In embodiments, a program that includes only microcurrent 707 (and not electroporation 706) is selected. In embodiments, a program that includes electroporation 706 and microcurrent 707 is selected. In this manner, in embodiments, one or more compounds of the composition comprises a large molecule, a small molecule, or both, and the program configures the control circuitry for transmission of electrical current based on the signal, e.g., received from the microchip of the reservoir.

Accordingly, in embodiments, the iontophoresis delivers a large molecule of the composition, a small molecule of the composition, or both, through a dermal layer of the portion of skin. In embodiments, the electrical current comprises a radiofrequency (RF) warming electrical current. In embodiments, the ultrasound vibrations create one or more microchannels in a dermal layer of the portion of skin. In embodiments, the ultrasound electrical current is transmitted at a frequency in the range of 100 kHz to 1 MHz.

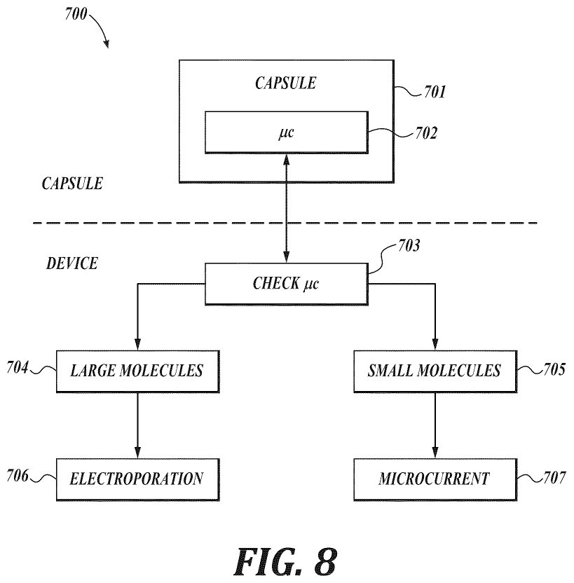

In another aspect, the disclosure provides a method for administering a composition to a portion of skin of a subject, the method comprising: receiving, with control circuitry of a system, a signal from a microchip of a reservoir of the system, wherein the reservoir contains at least a portion of the composition therein and the signal corresponds with one or more compounds of the composition in the reservoir; activating, with control circuitry of the system, an electromagnet of the system to move a contact-less piston of the system and displace at least the portion of the composition from the reservoir through an interior of a body of the system to an interior of an applicator capsule of the system for expulsion of at least the portion of the composition from one or more apertures of the applicator capsule; and transmitting, with control circuitry of the system, an electrical current through the portion of skin of the subject, wherein transmission of the electrical current is based on a program selected by the control circuitry based at least in part on the signal. As shown at FIG. 8 by way of a non-limiting example, a method 800 of using an application system of the disclosure to apply a composition to a portion of a subject's skin comprises, at step 801, a start step. At step 802, an applicator or reservoir filled with formula or composition is attached to the dispensing device comprising the body and other elements of the system; at step 803, the formula is identified by control circuitry of the system as it reads a contactless chip by way of receipt of the signal, for example, with a contactless reader (e.g., RFID reader); at step 804, generation of a magnetic field to move the piston and displace the composition from the reservoir; at step 805, dispense the composition from the system as the piston moves; at step 806, administer one or more microcurrent treatments; at step 807, apply formula with roller ball; and at step 808, finish.

In embodiments, an electrical impedance can be delivered as at least part of an ultrasound microcurrent for warming, which can range from about 10-50 kOhm. In embodiments, electrical pulses can be delivered in short bursts, for example, 1 msec burst for every 100 msec of time (1% load). In embodiments, a 1% load, a 2% load, a 3% load, a 4% load, a 5% load, a 6% load, a 7% load, an 8% load, a 9% load, 10% load, an 11% load, a 12% load, a 13% load, a 14% load, a 15% load, a 16% load, a 17% load, an 18% load, a 19% load, a 20% load, a 21% load, a 22% load, a 23% load, a 24% load, a 25% load, or a higher percentage load, can be applied for an ultrasound warming effect. Percentage load can be calculated by dividing the electrical burst time by the total time and multiplying the result by 100%. As would be understood by a person having ordinary skill in the art, a higher intensity ultrasound can be applied at a lower percentage load to avoid overheating the skin; similarly, a lower intensity ultrasound can be applied at a higher percentage load to ensure effective heating of the skin.

In embodiments, electroporation is delivered before, during, and/or after iontophoresis and/or ultrasound. In embodiments, iontophoresis is delivered before, during, and/or after electroporation and/or ultrasound. In embodiments, ultrasound is delivered before, during, and/or after electroporation and/or iontophoresis.

As an example method, one or more electrode pairs of the system can be operated in a concurrent mode, as follows:
Ultrasound ON for 90% load;
Ultrasound OFF, during which electroporesis and/or iontophoresis is applied.

As another example method, one or more electrode pairs of the system can be operated in a sequential mode, as follows:
Ultrasound ON for 50% load;
Ultrasound OFF, during which the skin cools enough to retain warming effect and pore opening;
Electroporesis and/or iontophoresis is applied.

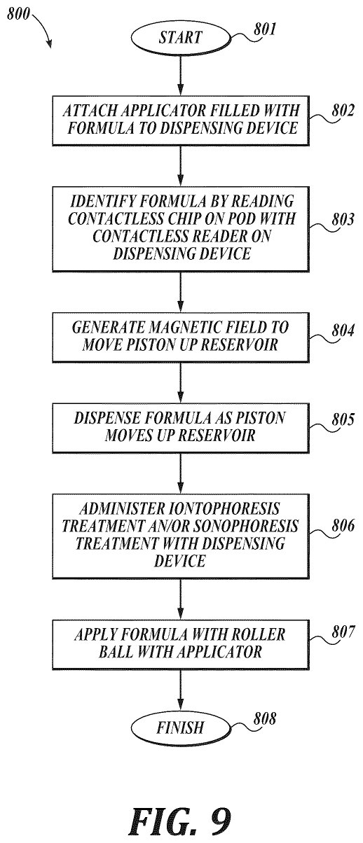

As shown at FIG. 9, an example applicator capsule 1000 is shown in an exploded configuration, and shows an example piston assembly. A capsule 1000 can include a cap 1105, a rollerball 1110, a fitment 1150, a body 1200, a piston 1250, a plug 1300, a push rod 1350, and an authentication chip 1500. The shown example piston assembly, and other structures shown for enabling movement of the composition within the capsule 1000, can be incorporated into various embodiments that include a rollerball and electrodes, as described herein. In embodiments, the cap 1105 can be disposed at a first end of the body 1200 and configured to attach to the body 1200. For example, the cap 105 can be threaded and twist tightened onto the body 1200 which can also be threaded (as shown) or the cap 1105 can be snap tightened onto the body 1200. The fitment 1150 can be disposed at the first end of the body 1200. The cap 1105, fitment 1150, body 1200, piston 1250, and plug 1300 can be fabricated from a polymer material. Non-limiting examples of materials for the cap 1105, fitment 1150, body 1200, piston 1250, and plug 1300 (either separately or together) can include a thermoplastic elastomer, polypropylene (PP), polyethylene terephthalate (PETG), acrylonitrile butadiene styrene (ABS), polycarbonate (PC), polyamide (Nylon), polystyrene (PS), low-density polyethylene (LDPE), high-density polyethylene (HDPE), or any combination thereof. For example, all pieces can be fabricated from PP. In another example, the cap 1105 can be fabricated from PP, the fitment 1150 can be fabricated from PETG, the body 1200 can be fabricated from PP, the piston 1250 can be fabricated from LDPE, and the plug 1300 can be fabricated from PP.

The body 1200 can be shaped substantially cylindrical and can include a first opening at the first end and a reservoir 1220 at a second end, wherein an inner diameter of the first opening is wider than an inner diameter of the reservoir 1220. Both the first opening and reservoir 1220 can be substantially annular. The first opening of the body 1200 can include a length of substantially straight stroke having the inner diameter of the first opening. The first opening can taper more narrowly down to the inner diameter of the reservoir 1220. The reservoir 1220 can be substantially straight and connected to the tapered portion extending from the first length of substantially straight stroke. It can be appreciated that the cross-sectional shape of the body 1200 can be fabricated as any of a myriad of other shapes, for example triangular, square, pentagonal, hexagonal, octagonal, or the like.

The first opening can be configured to hold the fitment 1150. The fitment 1150 can include an exterior shape that is configured to be push-fit into the first opening, wherein the fitment 1150 shape can contour to the length of substantially straight stroke and the tapered portion extending from the length of substantially straight stroke. Thus, the fitment 1150 can form a liquid-tight seal with the first end of the body 1200. In embodiments, the fitment 1150 can be fabricated as part of the body 1200 at the first end of the body 1200. For example, the fitment 1150 and body 1200 can be molded together as one piece. The fitment 1150 can be configured to hold the rollerball 1110, wherein an interior shape of the fitment 1150 is substantially hemispherical. A first end of the fitment 1150 can include a rollerball retainer 1170. The rollerball retainer 1170 can be an annular extrusion of material from the first end of the fitment 1150 that can slightly taper inwards towards the interior of the fitment 1150 such that the inner diameter of the rollerball retainer 1170 is narrower than the diameter of the rollerball 1110. The rollerball 1110 can be installed in the fitment 1150 by pushing the rollerball 1110 through the opening of the rollerball retainer 1170. The rollerball retainer 1170 can elastically deform outwards (i.e., the rollerball retainer 1170 opening widens and may thus be fabricated from a deformable polymer) to accommodate the rollerball 1110 when the rollerball 1110 is pushed through and then return to its original inner diameter. The rollerball 1110 can be fabricated from glass, metal, or a polymer, such as the ones described for the cap 1105, fitment 1150, body 1200, piston 1250, and plug 1300.

The piston 1250 and plug 1300 can be disposed at the second end of the body 1200. The piston 1250 can be shaped substantially disc-like and can include an outer diameter equal to, or marginally narrower than, the inner diameter of the reservoir 1220 such that a liquid-tight seal can be formed between the piston 1250 and an interior of the reservoir 1220. The plug 1300 can also be shaped substantially disc-like. The piston 1250 can be installed in the reservoir 1220 and the plug 1300 can be installed at the second end of the body 1200, wherein the plug 1300 prevents egress of the piston 1250. The plug 1300 can be push-fit, snap-fit, twist-tightened, or chemically attached to the second end of the body 1200. The plug 1300 can include a hole in the middle configured to allow the push rod 1350 to reversibly travel through. In another aspect, the plug 1300 can be fabricated as a part of the body 1200 at the second end of the body 1200. For example, the plug 1300 and body 1200 can be molded together as one piece. It can be appreciated that the piston 1250 and plug 1300 can be shaped according to the cross-sectional shape of the body 1200, and the disc-like shape is just one example.

A first end of the push rod 1350 can be configured to abut the piston 1250. For example, the piston 1250 can include a molded indentation opening towards the second end of the body 1200 having a shape complementary to the first end of the push rod 1350. A second end of the push rod 1350 (not shown) can be attached to a metering device (not shown) configured to translate the push rod 1350 a predetermined distance. The metering device can take the form of an applicator, and such an applicator would be configured to receive the capsule. The abutting of the first end of the push rod 1350 against the piston 1250 therefore causes the piston 1250 to travel towards the first end of the body (i.e., into the reservoir 1220) the same predetermined distance the push rod 1350 is translated.

The reservoir 1220 can be configured to hold a composition, e.g., a solution. In embodiments, the solution can be a cosmetic. In embodiments, the composition can comprise one or more small molecules to be delivered to and/or through the skin, one or more large molecules to be delivered to and/or through the skin, or any combination thereof.

In embodiments, the composition can be a topical medication, such as a serum, an ointment, a lotion, oil, an essential oil, a cream, a gel, a paste, foam, a water-based mixture, and an alcohol-based mixture (e.g., a tincture), or any combination thereof. The topical medication can include active ingredients, such as drug content, for treating skin ailments, and/or can include nutrients, for example, vitamins and minerals.

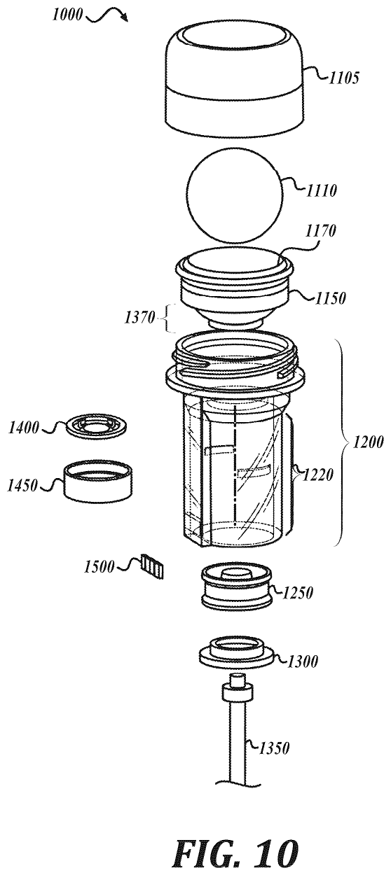

FIG. 10 illustrates a cross-sectional view schematic of the capsule 1000, as a non-limiting example. The fitment 1150 can include a well 1205. The well 1205 can be the volume between the rollerball 1110 and the interior of the fitment 1150. The well 1205 can be configured to receive a predetermined volume of solution from the reservoir 1220. The fitment 1150 can include a solution regulator 1137 disposed at a second end of the fitment 1150 through which the predetermined volume of solution is transferred from the reservoir 1220 to the well 1205. The solution regulator 1137 can be an orifice or a partially open orifice through which the solution flows towards the well 1205, wherein the solution regulator 1137 can be configured to meter the predetermined volume of solution passing through and preventing undesired reverse flow of solution from the well 1205 towards the reservoir 1220.

In embodiments, the solution regulator 1137 can be provided by a check valve 1400. The solution regulator 1137 can be substantially open and configured to allow attachment or insertion of the check valve 1400. The check valve 1400 can be installed inside or proximal to the second end of the fitment 1150 and held in place via a check valve holder 1450. The check valve 1400 and check valve holder 1450 can be installed in the reservoir 1220 through the second end of the body 1200. For example, the check valve 1400 can be installed first and the check valve holder 1450 can be installed after, wherein the check valve holder 1450 includes features that allow it to be snap fit into complementary features of the reservoir 1220. In another non-limiting example, the check valve 1400 can be coupled to the check valve holder 1450 prior to installation of both into the reservoir 1220. In another non-limiting example, the check valve 1400 and check valve holder 1450 can be chemically bonded to the reservoir 1220 by, for example, glue, epoxy, caulking, or any combination thereof. In embodiments, the check valve 1400 and check valve holder 1450 can be fabricated as a single part, i.e., the check valve 1400 includes features that allow it to be snap fit into the complementary features of the reservoir without requiring the separate check valve holder 1450. Non-limiting examples of materials for the check valve holder 1450 include a thermoplastic elastomer, PP, PETG, ABS, PC, Nylon, PS, LDPE, HDPE, and any combination thereof.

In embodiments, the check valve 1400 can be a one-way valve allowing solution transfer in a single direction of flow (or preventing solution transfer in said direction of flow when flow stoppage is desired). The check valve 1400 can be a deformable membrane held in position via tension, wherein the position in tension forms a liquid-tight seal. For example, the check valve 1400 can be fabricated from LDPE or PETG. In response to a force applied on the deformable membrane originating from a single direction, the membrane can deflect along the direction of the applied force. Upon release/ceasing of the applied force, the tension on the membrane can return the membrane to its un-deflected orientation. Thus, the check valve 1400 can be in one of two states. As shown in FIG. 10, a first state can be closed and liquid-tight, wherein the check valve 1400 does not allow solution from the reservoir 1220 to transfer to the well 1205. It can be appreciated by those in the art that other one-way valves may be used without departing from the scope and spirit of the disclosure, for example, a spring-ball construction.

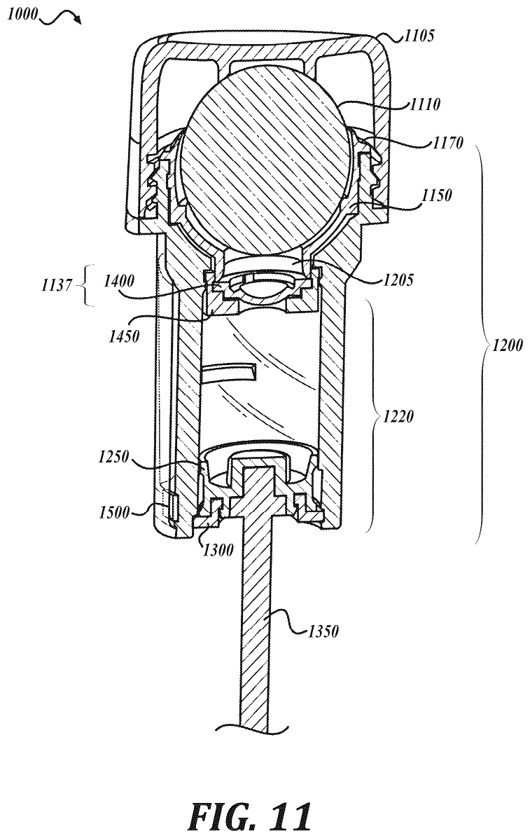

FIG. 11 illustrates a cross-sectional view schematic of the capsule 1000 during displacement of the push rod 1350, according to a non-limiting example. A second state of the check valve 1400 can be open, wherein the check valve 1400 membrane is deflected, thereby breaking the liquid-tight seal and allowing solution to transfer through the check valve 1400.

In embodiments, the push rod 1350 can be translated a predetermined distance. The push rod 1350 can concomitantly translate the piston 1250 the predetermined distance in the direction of the first end of the body 1200. Since the solution in the reservoir 1220 may not be compressible, the force of the piston 1250 pushing on the solution can result in the check valve 1400 switching from the first (closed) state to the second (open) state. The open check valve 1400 can then allow the predetermined volume of solution to transfer from the reservoir 1220 to the well 1205. The rollerball 1110 can be spherical and include a first portion of surface area in contact with the solution that was transferred to the well 1205. The rollerball 1110 can include a second portion of surface area exposed to the exterior and configured to contact a user's skin. The rollerball 1110 may be configured to roll across the user's skin and transfer the predetermined volume of solution, for example the topical medication, from the well 1205 to the user's skin. As the rollerball 1110 is rotated over the user's skin and deposits the solution onto the user's skin, the second portion of surface area rolls into the well 1205 and is coated again with more solution. Notably, the fitment 1150 can be fabricated to include some play between the interior of the fitment 1150 and the rollerball 1110 to allow ease of rolling of the rollerball 1110 and facilitate re-coating of the rollerball 1110 without the interior of the fitment 1150 scraping off said coating of solution as the rollerball 1110 rolls.

The predetermined distance the push rod 1135 is translated can be determined by calculating the distance needed for the piston 1250 to travel in order to displace the predetermined volume of solution in the reservoir 1220. The maximum predetermined volume of solution transferred from the reservoir 1220 can be determined by calculating the volume of solution the well 1205 is capable of holding. The predetermined volume of solution actually transferred from the reservoir 1220 to the well 1205 can be determined by the metering device, for example the user can be attempting to complete a recommended regimen for treating a skin ailment. Thus, the user may desire a specific dosage of topical medication for applying to the user's skin and the metering device can be configured to transfer the predetermined volume of solution from the reservoir 1220 to the well 1205 at a predetermined frequency. For example, the metering device can transfer 0.3 mL of solution on a daily basis during a 14-day treatment plan, wherein the metering device is configured to allow the user to apply the solution within a preset length of time, for example 3 minutes per day. An on-board chip (not shown) in the metering device can record the user's usage and a position of the piston 1250, wherein upon determining that the position of the piston 1250 correlates to a 14th day of the treatment, the metering device may notify the user to replace the capsule 1000. In response to determining that the user has removed the capsule, the metering device can adjust and reset the position of the piston 1250 to a position correlating to a start of the 14-day treatment plan. In addition, the metering device can reset the on-board chip to begin recording the user's usage again anew.

Advantageously, the built-in solution regulating feature, i.e., the check valve 1400, can prevent excess solution from transferring to the well 1205 once the piston 1250 stops and the release of force (and the tension on the check valve 1400) closes the check valve 1400. Therefore, this prevents the user from over-applying the solution, which can be important when the solution is a topical medication including a particular active ingredient, for example a drug, which should not be dosed in excess. Additionally, this may be aided by the metering device in which the capsule 1000 is installed, wherein the metering device prevents the user from overdosing by only translating the piston 1250 via the push rod 1350 a predetermined number of instances within a predetermined timeframe, for example once per day, and not more frequently than programmed regardless of user input (e.g., the user prompting the metering device for another dose).

### NON-LIMITING EMBODIMENTS

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.

Embodiment 1. A system for application of a composition to a portion of skin of a subject, the system comprising: an applicator capsule, comprising: a rollerball configured for a rolling contact with the portion of skin of the subject and a plurality of electrodes configured for a transmission of an electrical current through the portion of skin of the subject, a reservoir configured to hold and release the composition therefrom, and an ultrasound transducer configured to transmit ultrasound for sonophoresis; and a body, configured for reversible attachment of the applicator capsule thereto, and comprising a contact-less piston configured to expel the composition from the reservoir; wherein one or more apertures of the applicator capsule fluidly connect the interior of the applicator capsule to an exterior of the applicator capsule for delivery of the composition from the reservoir to the portion of skin.

Embodiment 2. The system of Embodiment 1 or any other Embodiment, further comprising control circuitry configured to electronically control the at least one operation of the contact-less piston.

Embodiment 3. The system of any one of Embodiments 1-2 or any other Embodiment, further comprising control circuitry operably connected to and configured to receive a signal from a microchip of the reservoir that corresponds with one or more compounds of the composition, wherein the control circuitry electronically controls the transmission of the electrical current, based on a program selected by the control circuitry based at least in part on the signal, for delivery of the one or more compounds to the portion of skin of the subject.

Embodiment 4. The system of any one of Embodiments 1-3 or any other Embodiment, wherein the one or more compounds of the composition comprises a large molecule, a small molecule, or both, and wherein the program configures the control circuitry for the transmission of the electrical current based on the signal.

Embodiment 5. The system of any one of Embodiments 1˜4 or any other Embodiment, wherein the contact-less piston is responsive to an activation of an electromagnet and the at least one operation comprises activation of the electromagnet for movement of the contact-less piston and displacement of the composition from the formula pod.

Embodiment 6. The system of any one of Embodiments 1-5 or any other Embodiment, wherein the electrical current comprises electroporesis of at least one compound of the composition to the portion of skin.

Embodiment 7. The system of any one of Embodiments 1-6 or any other Embodiment, wherein the electrical current comprises iontophoresis of at least one compound of the composition through the portion of skin.

Embodiment 8. The system of any one of Embodiments 1-7 or any other Embodiment, wherein the iontophoresis delivers a large molecule of the composition, a small molecule of the composition, or both, through a dermal layer of the portion of skin.

Embodiment 9. The system of any one of Embodiments 1-8 or any other Embodiment, wherein the electrical current activates the ultrasound transducer for sonophoresis.

Embodiment 10. The system of any one of Embodiments 1-9 or any other Embodiment, wherein the sonophoresis creates one or more microchannels in a dermal layer of the portion of skin.

Embodiment 11. The system of any one of Embodiments 1-10 or any other Embodiment, wherein the ultrasound for sonophoresis is transmitted at a frequency in the range of 100 kilohertz to 1 megahertz.

Embodiment 12. A method for administering a composition to a portion of skin of a subject, the method comprising: activating, with control circuitry of the system, an electromagnet of the system to move a contact-less piston of the system and displace at least the portion of the composition from a reservoir for expulsion of at least the portion of the composition from one or more apertures of the applicator capsule, responsive to receiving, with control circuitry of the system, a signal from a microchip of a reservoir of the system, wherein the reservoir contains at least a portion of the composition therein and the signal corresponds with one or more compounds of the composition in the reservoir; and transmitting, with control circuitry of the system, an electrical current through the portion of skin of the subject, wherein transmission of the electrical current is based on a program selected by the control circuitry based at least in part on the signal.

Embodiment 13. The method of Embodiment 12 or any other Embodiment, wherein the electrical current comprises electroporesis of at least one compound of the composition to the portion of skin.

Embodiment 14. The method of any one of Embodiments 12-13 or any other Embodiment, wherein the electrical current comprises iontophoresis of at least one compound of the composition through the portion of skin.

Embodiment 15. The method of any one of Embodiments 12-14 or any other Embodiment, wherein electrical current activates an ultrasound transducer for sonophoresis.

While illustrative embodiments have been illustrated and described, it will be appreciated that various changes can be made therein without departing from the spirit and scope of the invention.

The embodiments of the invention in which an exclusive property or privilege is claimed are defined as follows:

1. A system for application of a composition to a portion of skin of a subject, the system comprising:
an applicator capsule, comprising: a rollerball configured for a rolling contact with the portion of skin of the subject and a plurality of electrodes configured for a transmission of an electrical current through the portion of skin of the subject, a reservoir configured to hold and release the composition therefrom, and an ultrasound transducer configured to transmit ultrasound for sonophoresis; and
a body, configured for reversible attachment of the applicator capsule thereto, and comprising a contact-less piston configured to expel the composition from the reservoir;
wherein one or more apertures of the applicator capsule fluidly connect the interior of the applicator capsule to an exterior of the applicator capsule for delivery of the composition from the reservoir to the portion of skin.

2. The system of claim 1, further comprising control circuitry configured to electronically control the at least one operation of the contact-less piston.

3. The system of claim 1, further comprising control circuitry operably connected to and configured to receive a signal from a microchip of the reservoir that corresponds with one or more compounds of the composition, wherein the control circuitry electronically controls the transmission of the electrical current, based on a program selected by the control circuitry based at least in part on the signal, for delivery of the one or more compounds to the portion of skin of the subject.

4. The system of claim 3, wherein the one or more compounds of the composition comprises a large molecule, a small molecule, or both, and wherein the program configures the control circuitry for the transmission of the electrical current based on the signal.

5. The system of claim 1, wherein the contact-less piston is responsive to an activation of an electromagnet and the at least one operation comprises activation of the electromagnet for movement of the contact-less piston and displacement of the composition from the formula pod.

6. The system of claim 1, wherein the electrical current comprises electroporesis of at least one compound of the composition to the portion of skin.

7. The system of claim 1, wherein the electrical current comprises iontophoresis of at least one compound of the composition through the portion of skin.

8. The system of claim 7, wherein the iontophoresis delivers a large molecule of the composition, a small molecule of the composition, or both, through a dermal layer of the portion of skin.

9. The system of claim 1, wherein the electrical current activates the ultrasound transducer for sonophoresis.

10. The system of claim 9, wherein the sonophoresis creates one or more microchannels in a dermal layer of the portion of skin.

11. The system of claim 10, wherein the ultrasound for sonophoresis is transmitted at a frequency in the range of 100 kilohertz to 1 megahertz.

12. A method for administering a composition to a portion of skin of a subject, the method comprising:
activating, with control circuitry of the system, an electromagnet of the system to move a contact-less piston of the system and displace at least the portion of the composition from a reservoir for expulsion of at least the portion of the composition from one or more apertures of the applicator capsule, responsive to receiving, with control circuitry of the system, a signal from a microchip of a reservoir of the system, wherein the reservoir contains at least a portion of the composition therein and the signal corresponds with one or more compounds of the composition in the reservoir; and
transmitting, with control circuitry of the system, an electrical current through the portion of skin of the subject, wherein transmission of the electrical current is based on a program selected by the control circuitry based at least in part on the signal.

13. The method of claim 12, wherein the electrical current comprises electroporesis of at least one compound of the composition to the portion of skin.

14. The method of claim 12, wherein the electrical current comprises iontophoresis of at least one compound of the composition through the portion of skin.

15. The method of claim 12, wherein electrical current activates an ultrasound transducer for sonophoresis.
